(19) United States
(12) Patent Application Publication
(10) Pub. No.: US 2017/0086689 A1
(43) Pub. Date: Mar. 30, 2017

(54) ELECTRONIC DEVICE INCLUDING AMBIENT LIGHT COMPENSATION CIRCUIT FOR HEART RATE GENERATION AND RELATED METHODS

(71) Applicant: Apple Inc., Cupertino, CA (US)

(72) Inventors: Tao SHUI, Cupertino, CA (US); Dong ZHENG, Los Altos, CA (US)

(21) Appl. No.: 14/870,524

(22) Filed: Sep. 30, 2015

(51) Int. Cl. A61B 5/024; A61B 5/00

(52) U.S. Cl. CPC A61B 5/02433; A61B 5/7278; A61B 5/02438; A61B 5/681; A61B 5/6898; A61B 5/0022; A61B 5/742; A61B 5/7475; A61B 5/7225; A61B 2560/0247; A61B 2560/0475; A61B 2562/0238

(57) ABSTRACT

An electronic device may include an optical source capable of supplying light to an adjacent user's body part having blood flow therein and an optical sensor capable of sensing light from the user's body part. The electronic device may also include at least one gain stage coupled to the optical sensor and a compensation circuit coupled to the at least one gain stage and capable of generating a compensated output signal. The compensation circuit may include a memory capable of storing ambient light compensation data, and a digital-to-analog converter (DAC) coupled to the at least one gain stage and capable of compensating for ambient light based upon the stored ambient light compensation data. The electronic device may also include a processor capable of generating a user's heart rate based upon the compensated output signal.

### TECHNICAL FIELD

The present invention relates to the field of electronics, and, more particularly, to optical sensing devices for heart rate generation and related methods.

### BACKGROUND

As mobile wireless communications devices become increasingly popular, so does the desire for the devices to become smaller while increasing functionality. One form of mobile wireless communications device is a wearable mobile wireless communications device. For example, a wearable mobile wireless communications device may be in the form of jewelry or a watch.

A wearable mobile wireless communications device, for example, in the form of a watch, may provide a user with different types of data, which may not be limited to only providing the user with the time of day. For example, a wearable mobile wireless communications device may provide notifications to the user, such as email messages, reminders, etc. The notifications may be visually displayed, or in some cases, the notifications may be provided through haptic feedback.

Many wearable mobile wireless communications devices also provide the user with information related to the health and activity status of the user. For example, the wearable device may provide the user with movement information (i.e., steps walked, distance traveled, flights climbed, etc.) and certain biometric data, such as, for example, heart rate.

### SUMMARY

An electronic device may include an optical source capable of supplying light to an adjacent user's body part having blood flow therein and an optical sensor capable of sensing light from the user's body part. The electronic device may also include at least one gain stage coupled to the optical sensor and a compensation circuit coupled to the at least one gain stage and capable of generating a compensated output signal. The compensation circuit may include a memory capable of storing ambient light compensation data, and a digital-to-analog converter (DAC) coupled to the at least one gain stage and capable of compensating for ambient light based upon the stored ambient light compensation data. The electronic device may also include a processor capable of generating a user's heart rate based upon the compensated output signal. Accordingly, ambient light that may be sensed via the optical sensor may be compensated, for example, to generate a more accurate heart rate of the user.

The compensation circuit may also include a filter coupled to the memory and having at least one filter coefficient based upon a gain of the at least one gain stage. The memory may be capable of storing ambient light compensation data to account for ambient light interference errors, for example.

The optical source may include at least one light emitting diode, for example. The optical source may include at least one infrared light source.

The at least one gain stage may be capable of generating a sensed light output having a sensed light component and an ambient light component. The DAC may be capable of compensating for the ambient light by subtracting the ambient light component from the sensed light component based upon the stored ambient light compensation data, for example.

The electronic device may also include an analog-to-digital converter (ADC) coupled to the at least one gain stage and the DAC. The processor may be capable of determining whether the optical source is adjacent the user's body part based upon the compensated output signal, for example.

The at least one gain stage may include a plurality of gain stages. The at least one gain stage may include at least one amplifier.

A method aspect is directed to a method of generating a user's heart rate using an electronic device that includes an optical source capable of supplying light to an adjacent user's body part having blood flow therein, an optical sensor capable of sensing light from the user's body part, and at least one gain stage coupled to the optical sensor. The method may include using a compensation circuit coupled to the at least one gain stage to generate a compensated output signal by storing in a memory ambient light compensation data, and using a digital-to-analog converter (DAC) coupled to the at least one gain stage to compensate for ambient light based upon the stored ambient light compensation data. The method also includes using a processor to generate the user's heart rate based upon the compensated output signal.

### BRIEF DESCRIPTION OF THE DRAWINGS

FIG. 1 is a top view of an electronic device being worn by a user according to an embodiment.

FIG. 2 is a schematic block diagram of the electronic device of FIG. 1

FIG. 3 is a bottom view of a portion of the electronic device of FIG. 1.

FIG. 4 is a schematic circuit equivalent diagram of a portion of the electronic device of FIG. 1

### DETAILED DESCRIPTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

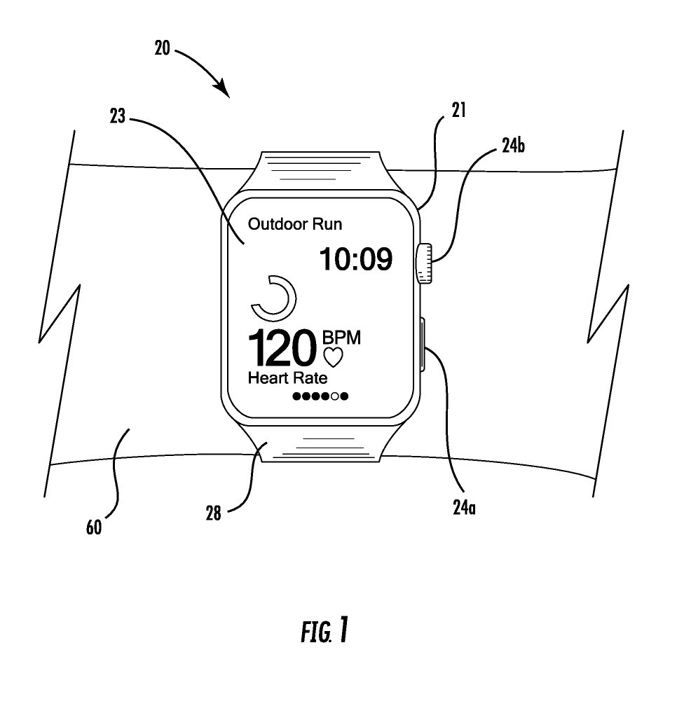

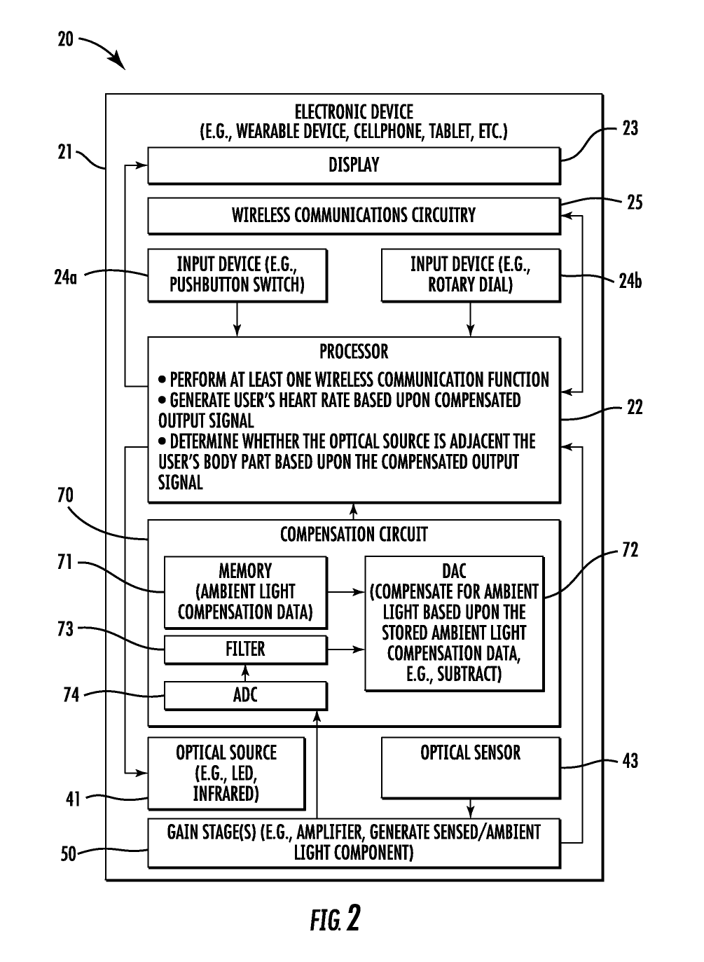

Referring initially to FIGS. 1 and 2, an electronic device 20 illustratively includes a device housing 21 and a processor 22 carried by the device housing. The electronic device 20 is illustratively a mobile wireless communications device, for example, a wearable wireless communications device, and includes a band 28 or strap for securing it to a user. The electronic device 20 may be another type of electronic device, for example, a cellular telephone, a tablet computer, a laptop computer, etc.

Wireless communications circuitry 25 (e.g. cellular, WLAN Bluetooth, etc.) is also carried within the device housing 21 and coupled to the processor 22. The wireless communications circuitry 25 cooperates with the processor 22 to perform at least one wireless communications function, for example, for voice and/or data. In some embodiments, the electronic device 20 may not include wireless communications circuitry 25.

A display 23 is also carried by the device housing 21 and is coupled to the processor 22. The display 23 may be a liquid crystal display (LCD), for example, or may be another type of display, as will be appreciated by those skilled in the art.

Finger-operated user input devices 24a, 24b, illustratively in the form of a pushbutton switch and a rotary dial are also carried by the device housing 21 and is coupled to the processor 22. The pushbutton switch 24a and the rotary dial 24b cooperate with the processor 22 to perform a device function in response to operation thereof. For example, a device function may include a powering on or off of the electronic device 20, initiating communication via the wireless communications circuitry 25, and/or performing a menu function. The processor 22 may also generate a heart rate of the user or other user data, as will be explained in further detail below.

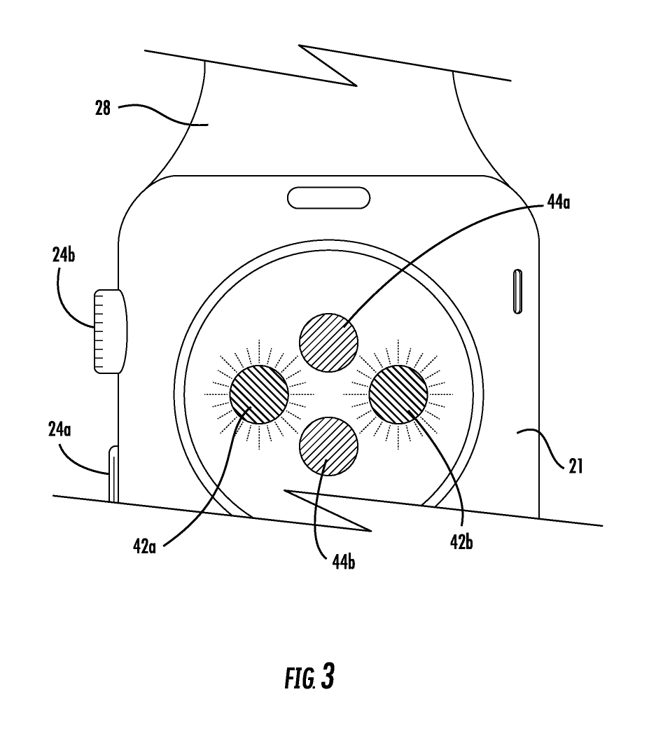

Referring additionally to FIG. 3, the electronic device 20 illustratively includes an optical source 41 that supplies supplying light to an adjacent user's body part 60 having blood flow therein, for example, the user's wrist. The optical source 41 illustratively includes light emitting diodes (LEDs) 42a, 42b carried by respective openings in the device housing 21 and that emit light in the visible spectrum, particularly green, and the infrared spectrum. Of course, the LEDs 42a, 42b may emit light in different spectrums, and while two LEDs are illustrated, it will be appreciated that there may be any number of LEDs.

An optical sensor 43 senses light from the user's body part 60. The optical sensor 43 illustratively includes a pair of photodiodes 44a, 44b that are also carried by respective openings in the device housing 21 and that may be responsive to light in both visible and infrared spectrums, as will be appreciated by those skilled in the art. While two photodiodes 44a, 44b are illustrated, it will be appreciated that there may be any number of photodiodes.

The processor 22 cooperates with the optical sensor and the optical source to generate a heart rate of the user. As will be appreciated by those skilled in the art, the heart rate is generated based upon photoplethysmography. Photoplethysmography is based on the fact that blood is red because it reflects red light and absorbs green light. Accordingly, the LEDs 42a, 42b, emit green light and cooperate with the photodiodes 44a, 44b to detect the amount of blood flowing through the user's body, i.e., the user's wrist, at any given moment. When the user's heart beats, the blood flows in the user's body, and the green light absorption, is greater. Between beats, the blood flows, and consequently, the green light absorption, is less. The processor 22 may flash the LEDs 42a, 42b rapidly, for example, hundreds of times per second, which may used for calculating the number of times the heart beats each minute, i.e., the heart rate.

The processor 22 may also be capable of determining whether the optical source 41 is adjacent the user's body part 60 based upon the compensated output signal. This may be particularly advantageous for determining whether the electronic device 20 is on or off the user 60. For example, where the electronic device 20 is a wrist watch, the processor 22 may cooperate with the optical sensor 43 to determine whether the electronic device is on or off the user's wrist. In some embodiments, heart rate calculation or other optical source 41 or optical sensor 43 based operations will not be performed, i.e., will indicate an error, unless the electronic device 20 is determined as being worn or on the user's wrist, for example.

The processor 22 may also compensate for low signal levels by either or both of increasing brightness of the LEDs 42a, 42b and the sampling rate of the photodiodes 44a, 44b. However, ambient light, for example, daylight, may affect the generation of the heart rate. Accordingly, it may be desirable to compensate for this ambient light.

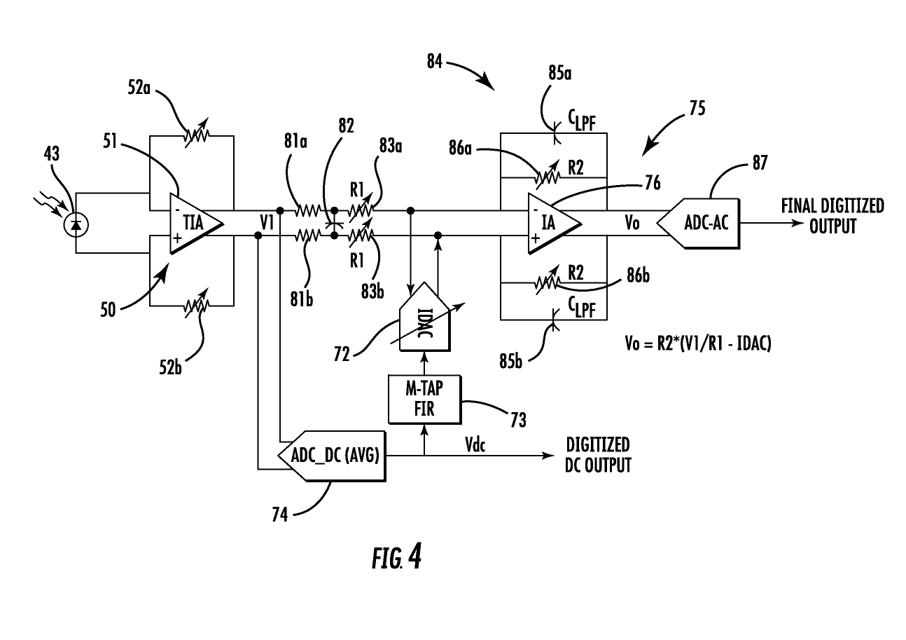

Referring now additionally to FIG. 4, a schematic diagram of a portion of the electronic device is illustrated. A first gain stage 50 is coupled to the optical sensor 43. The first gain stage 50 illustratively includes an amplifier 51, for example, a transimpedance amplifier (TIA). The first gain stage 50 outputs a sensed light output signal based upon the optical sensor 43. The sensed light output signal includes a sensed light component and an ambient light component. Feedback loops that respectively include variable resistors 52a, 52b are coupled between respective inputs and outputs of the amplifier 51.

A compensation circuit 70 is coupled to the first gain stage 50. The compensation circuit 70 generates a compensated output signal. The compensation circuit 70 also includes a memory 71 that stores ambient light compensation data to account for ambient light interference errors. The compensation circuit 70 also includes a digital-to-analog converter (DAC) 72 coupled to the first gain stage for compensating for ambient light based upon the stored ambient light compensation data.

The compensation circuit 70 also includes a filter 73 coupled to the memory 71. The filter 73, which may be M-tap weighted moving average (WMA) digital filter, has filter coefficients that are based upon a gain of the gain stage 50. The filter 73 cooperates with the DAC 72 to subtract the ambient light component from the sensed light output signal, thus leaving the sensed light component.

The compensation circuit 70 also includes first analog-to-digital converter (ADC) 74 coupled between the output of the first gain stage 50 and the filter 73. The first ADC 74 samples the output of the first gain stage 50 and feeds this to the filter 73.

A second gain stage 75 is coupled to the DAC 72 and the output of the first gain stage 50. The second gain stage 75 may be in the form of an amplifier, for example. Illustratively, a pair of resistors 81a, 81b, a coupling capacitor 82, and a pair of variable resistors 83a, 83b are coupled between the second gain stage 75 and the first gain stage 50 and coupled to the DAC 72.

A low-pass filter circuit 84 is coupled to the second gain stage 75. The low-pass filter circuit 84 includes parallel coupled first and second capacitors 85a, 85b and resistors 86a, 86b.

A second analog-to-digital converter (ADC) 87 is coupled to the output of the second gain stage 75. The output of the second ADC 87 is the compensated output signal.

It should be noted that in some embodiments, the first and second ADCs 74, 87, the filter 73, the second gain stage 75, and the DAC 72 may be carried by or on a integrated circuit, for example, a programmable gate array (PGA), while the first gain stage 50, and the optical sensor 43 may be off of or remote from the integrated circuit. Of course, some other arrangement of or all of the components described herein may be carried by one or more integrated circuits.

Further details of the electronic device 20 and its operation with respect to the compensation circuit 70 will now be described. The compensation circuit 70 may be considered a DC cancellation loop employing a digital weighted moving average (WMA) filter, i.e., the filter 73. The compensation circuit 70 uses a time-multiplexed ambient cancellation scheme, in which the first ADC 74 is a relatively fast and medium resolution ADC, the filter 73 is an M-tap WMA, the DAC 72 is a low-noise current-mode DAC, the second gain stage 75 includes a programmable-gain instrumentation amplifier (IA) 76, and the second ADC 87 is a high resolution ADC.

Before the “actual” signal (i.e., signal from the optical sensor used to generate the user's heart rate) is applied, the first ADC 74 takes multiple snap-shots on the output of the TIA amplifier 51 defining the first gain stage 50, denoted as V_{dc}. A local average may be calculated to increase conversion resolution. V_{dc} includes ambient light interference information which is subtracted out through the cancellation loop when the “actual” signal is applied at the input of the first amplifier 51.

The filter 73 may be considered a programmable M-tap WMA digital filter that can be used to further processes V_{dc} based on previous samples of the first ADC 74 before those signals are provided to the DAC 72. The number of taps, M, ranges from 1 to 8, which may depend on the activated measurement time slots. The coefficient of the WMA {a₀, a₁, . . . a_{M-1}} corresponds to the inverse of the external gain of the TIA amplifier 51 in current and previous time slots. These filter coefficients are be shifted in time together with the shifted data. The register memory of the filter 73 may have reset or non-reset at the beginning of each scan period. This may help to track the ambient information over multiple measurement scan periods for increased ambient light cancellation, for example.

In some embodiments, when the filter 73 is disabled, V_{dc} includes instantaneous ambient light information before the actual signal applied. When filter 73 is enabled, V_{dc} includes the most recent and previous M−1 samples of ambient light information before the actual signal is applied.

The M-tap coefficients of the filter 73 {a₀, a₁, . . . , a_{M-1}} can be calculated in the following way: denote the first time slot gain of the TIA amplifier 51 as G₁, the second time slot TIA gain as G₂, the third as G₃, and so on. Set a₀=1, then a₁=G₁/G₂, a₂=G₁/G₃, . . . , a_{M-1}=G₁/G_{M}, where G_{M} is the last time slot TIA gain. The gain of the TIA amplifier 51 for each time slot can be determined by corresponding registers. An internal lookup table can be implemented so that the gain ratio can be calculated and stored initially. Either M-tap averaged output or the pre-filtered V_{dc} can be sent to a host for system level algorithmic decision (e.g., similar to TIA and PGA gain adjustment).

V_{dc} is applied to an analog current domain through the DAC, which is in the form of a current mode DAC (IDAC) 72, so that during the normal measurement period, the ambient information can be subtracted from the incoming signal V₁ so that the wanted AC signal at time slot n can be amplified such that:

Vo(n)=R2*(V1/R1−V_{dc}/aₙ/Rdac)

where Rdac is the equivalent resistance of the IDAC 72, and aₙ is the current time slot normalized gain of the TIA amplifier 51.

In another embodiment, as will be appreciated by those skilled in the art, the first gain stage 50 may include, instead of the TIA amplifier 51, a resettable integrator, comparator, and a current source. This arrangement may reduce the relatively large DC common voltage so that the residual analog signal may be processed by the ADC 74.

Advantageously, the arrangement described above including the compensation circuit 70 may be particularly advantageous as it may reduce cost of manufacturing and increase performance. More particularly, an external AC coupling capacitor, which may add to form factor, size, and cost, may not be needed. Additionally, a relatively ultra-dynamic range ADC, which may use relatively more power and have a higher cost, may also not be needed. Performance may be increased due to reduced external interference.

A method aspect is directed to a method of generating a user's heart rate using the electronic device 20. The method includes using the compensation circuit 70 to generate a compensated output signal by storing in the memory 71 ambient light compensation data, and using the DAC 72 to compensate for ambient light based upon the stored ambient light compensation data. The method also includes using the processor 22 to generate the user's heart rate based upon the compensated output signal.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. An electronic device comprising:
an optical source capable of supplying light to an adjacent user's body part having blood flow therein;
an optical sensor capable of sensing light from the user's body part;
at least one gain stage coupled to the optical sensor;
a compensation circuit coupled to the at least one gain stage and capable of generating a compensated output signal, the compensation circuit comprising
a memory capable of storing ambient light compensation data, and
a digital-to-analog converter (DAC) coupled to the at least one gain stage and capable of compensating for ambient light based upon the stored ambient light compensation data; and
a processor capable of generating a user's heart rate based upon the compensated output signal.

2. The electronic device of claim 1 wherein the compensation circuit further comprises a filter coupled to the memory and having at least one filter coefficient based upon a gain of the at least one gain stage.

3. The electronic device of claim 1 wherein the memory is capable of storing ambient light compensation data to account for ambient light interference errors.

4. The electronic device of claim 1 wherein the optical source comprises at least one light emitting diode.

5. The electronic device of claim 1 wherein the optical source comprises at least one infrared light source.

6. The electronic device of claim 1 wherein the at least one gain stage is capable of generating a sensed light output having a sensed light component and an ambient light component.

7. The electronic device of claim 6 wherein the DAC is capable of compensating for the ambient light by subtracting the ambient light component from the sensed light component based upon the stored ambient light compensation data.

8. The electronic device of claim 1 further comprising an analog-to-digital converter (ADC) coupled to the at least one gain stage and the DAC.

9. The electronic device of claim 1 wherein the processor is capable of determining whether the optical source is adjacent the user's body part based upon the compensated output signal.

10. The electronic device of claim 1 wherein the at least one gain stage comprises a plurality of gain stages.

11. The electronic device of claim 1 wherein the at least one gain stage comprises at least one amplifier.

12. An electronic device comprising:
a housing;
wireless communications circuitry carried by the housing;
an optical source carried by the housing and capable of supplying light to an adjacent user's body part having blood flow therein;
an optical sensor carried by the housing and capable of sensing light from the user's body part;
at least one gain stage coupled to the optical sensor;
a compensation circuit coupled to the at least one gain stage and capable of generating a compensated output signal, the compensation circuit comprising
a memory capable of storing ambient light compensation data, and
a digital-to-analog converter (DAC) coupled to the at least one gain stage and capable of compensating for ambient light based upon the stored ambient light compensation data; and
a processor capable of generating a user's heart rate based upon the compensated output signal and cooperating with the wireless communications circuitry to perform at least one wireless communications function.

13. The electronic device of claim 12 wherein the compensation circuit further comprises a filter coupled to the memory and having at least one filter coefficient based upon a gain of the at least one gain stage.

14. The electronic device of claim 12 wherein the memory is capable of storing ambient light compensation data to account for ambient light interference errors.

15. The electronic device of claim 12 wherein the optical source comprises at least one light emitting diode.

16. The electronic device of claim 12 wherein the optical source comprises at least one infrared light source.

17. A method of generating a user's heart rate using an electronic device comprising an optical source capable of supplying light to an adjacent user's body part having blood flow therein, an optical sensor capable of sensing light from the user's body part, and at least one gain stage coupled to the optical sensor, the method comprising:
using a compensation circuit coupled to the at least one gain stage to generate a compensated output signal by
storing in a memory ambient light compensation data, and
using a digital-to-analog converter (DAC) coupled to the at least one gain stage to compensate for ambient light based upon the stored ambient light compensation data; and
using a processor to generate the user's heart rate based upon the compensated output signal.

18. The method of claim 17 wherein the compensation circuit further comprises a filter coupled to the memory and having at least one filter coefficient based upon a gain of the at least one gain stage.

19. The method of claim 17 wherein the memory stores ambient light compensation data to account for ambient light interference errors.

20. The method of claim 17 wherein the optical source comprises at least one light emitting diode.

21. The method of claim 17 wherein the optical source comprises at least one infrared light source.

22. The method of claim 17 wherein the at least one gain stage generates a sensed light output having a sensed light component and an ambient light component; and wherein the DAC compensates for the ambient light by subtracting the ambient light component from the sensed light component based upon the stored ambient light compensation data.

23. The method of claim 17 wherein using the processor further comprises using the processor to determine whether the optical source is adjacent the user's body part based upon the compensated output signal.
